(19) United States
(12) Patent Application Publication
(10) Pub. No.: US 2022/0301268 A1
(43) Pub. Date: Sep. 22, 2022

(54) DEVICES AND METHODS FOR REGISTERING AN IMAGING MODEL TO AN AUGMENTED REALITY SYSTEM BEFORE OR DURING SURGERY

(71) Applicant: 3D SYSTEMS INC., Rock Hill, SC (US)

(72) Inventors: Roy Porat, Tel Aviv (IL); Ran Bronstein, Modi'in (IL)

(73) Assignee: 3D SYSTEMS INC., Rock Hill, SC (US)

(21) Appl. No.: 17/696,386

(22) Filed: Mar. 16, 2022

Related U.S. Application Data
(60) Provisional application No. 63/162,628, filed on Mar. 18, 2021.

(51) Int. Cl. G06T 19/00; G06T 7/10; G02B 27/01; A61B 34/20; A61B 34/10; A61B 34/00

(52) U.S. Cl. CPC G06T 19/006; G06T 7/10; G02B 27/017; A61B 34/20; A61B 34/10; A61B 34/25; A61B 2034/107; A61B 2034/2074

(57) ABSTRACT

Systems and methods are provided for improving registration of AR (augmented reality) units at a surgery scene. Systems comprise augmented reality (AR) unit(s) comprising and/or in communication with head mounted display(s) (HMDs) used by a user, and physical device(s) made of sterilizable biocompatible material and configured as a registration template. The AR unit and/or segmentation software associated therewith may be configured to align a device representation of the physical device onto an imaging model of a patient, and the AR unit and/or the HMD may be configured to register, on the HMD, the device representation with the aligned imaging model onto the physical device, which is positioned with respect to the patient and is viewed through the HMD—to display the imaging model or parts thereof in a corresponding spatial relation to the patient. Registration using the physical device simplifies the coordination among real and virtual devices.

### CROSS-REFERENCE TO RELATED APPLICATIONS

This Application claims benefit of prior US Provisional Patent Application 63/162,628, filed on Mar. 18, 2021, entitled “DEVICES AND METHODS FOR REGISTERING AN IMAGING MODEL TO AN AUGMENTED REALITY SYSTEM BEFORE OR DURING SURGERY”, which is hereby incorporated by reference in its entirety.

### BACKGROUND OF THE INVENTION

### 1. Technical Field

The present invention relates to the field of AR (augmented reality) assisted surgery, and more particularly, to improving registration in AR surgical systems.

### 2. Discussion of Related Art

Prior techniques include three dimensional and two-dimensional (3D/2D) registration methods with respect to image modality, image dimensionality, registration basis, geometric transformation, user interaction, optimization procedure, subject, and object of registration.

Prior techniques include AR-based surgical navigation systems (AR-SNS) that use an optical see-through HMD (head-mounted display). The calibration of instruments, registration, and the calibration of the HMD are used to align the 3D virtual critical anatomical structures in the head-mounted display with the actual structures of the patient in the real-world scenario during the intra-operative motion tracking process.

Prior techniques include projecting a computerized tomography (CT) scan with Microsoft® Hololens® (Hololens) and then aligning that projection to a set of fiduciary markers.

Prior techniques include evaluations of the surgical accuracy of holographic pedicle screw navigation by head-mounted device using 3D intraoperative fluoroscopy.

### SUMMARY OF THE INVENTION

The following is a simplified summary providing an initial understanding of the invention. The summary does not necessarily identify key elements nor limit the scope of the invention, but merely serves as an introduction to the following description.

One aspect of embodiments of the present invention provides a system comprising: an augmented reality (AR) unit comprising and/or in communication with a head mounted display (HMD) used by a user, and a physical device made of sterilizable biocompatible material and configured as a registration template, wherein the AR unit and/or segmentation software associated therewith is configured to align a device representation of the physical device onto an imaging model of a patient, and wherein the AR unit and/or the HMD is configured to register, on the HMD, the device representation with the aligned imaging model onto the physical device, which is positioned with respect to the patient and is viewed through the HMD—to display the imaging model or parts thereof in a corresponding spatial relation to the patient.

One aspect of embodiments of the present invention provides a method comprising: aligning a device representation of a physical device onto an imaging model of a patient, and registering, on a HMD, the device representation with the aligned imaging model onto the physical device as positioned with respect to the patient and as viewed through the HMD—to display the imaging model or parts thereof in a corresponding spatial relation to the patient on the HMD.

These, additional, and/or other aspects and/or advantages of the present invention are set forth in the detailed description which follows: possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIG. 1A is a high-level schematic illustration of an operation scene with a registration device, according to some embodiments of the invention.

FIG. 1B is a high-level schematic illustration of using the registration device, according to some embodiments of the invention.

FIG. 1C is a high-level schematic block diagram of a registration system, according to some embodiments of the invention.

FIGS. 1D-F are high-level schematic block diagrams of AR, units and related HMDs, according to some embodiments of the invention.

FIGS. 2A-2F include high-level schematic illustrations of physical devices, according to some embodiments of the invention.

FIGS. 3A, 3B and 4 are high-level flowcharts illustrating methods, according to some embodiments of the invention.

FIG. 5 is a high-level block diagram of an exemplary computing device, which may be used with embodiments of the present invention.

### DETAILED DESCRIPTION OF THE INVENTION

In the following description, various aspects of the present invention are described. For purposes of explanation, specific configurations and details are set forth in order to provide a thorough understanding of the present invention. However, it will also be apparent to one skilled in the art that the present invention may be practiced without the specific details presented herein. Furthermore, well known features may have been omitted or simplified in order not to obscure the present invention. With specific reference to the drawings, it is stressed that the particulars shown are by way of example and for purposes of illustrative discussion of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments that may be practiced or carried out in various ways as well as to combinations of the disclosed embodiments. Also, it is to be understood that the phraseology and terminology employed herein are for the purpose of description and should not be regarded as limiting.

Unless specifically stated otherwise, as apparent from the following discussions, it is appreciated that throughout the specification discussions utilizing terms such as “processing”, “computing”, “calculating”, “determining”. “enhancing”, “deriving” or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

Embodiments of the present invention provide efficient and economical methods and mechanisms for registering a patient imaging model in an augmented reality (AR) surgery system and may thereby provide improvements to the technological field of AR-assisted surgery. Various embodiments comprise using a registration template for registering the patient imaging model in the AR surgery system before and/or during surgery. Embodiments may include systems and methods for improving registration of AR (augmented reality) units at a surgery scene. Some embodiments include AR unit(s) that include and/or are in communication with head mounted display(s) (HMDs) used by a user, and physical device(s) made of sterilizable biocompatible material and configured as a registration template. The AR unit and/or segmentation software associated therewith may be configured to align a device representation of the physical device onto an imaging model of a patient, and the AR unit and/or the HMD may be configured to register, on the HMD, the device representation with the aligned imaging model onto the physical device, which is positioned with respect to the patient and is viewed through the HMD—to display the imaging model or parts thereof in a corresponding spatial relation to the patient. Embodiments may include methods to coordinate the design of the physical devices and provide for spatially synchronizing virtual model(s) based e.g., on imaging data with the actual patient, and with additional displays associated with the operation. Registration using the physical device simplifies the coordination among real and virtual devices.

In current AR-assisted surgery systems, a patient imaging model (e.g., derived from past images or recent CT or magnetic resonance imaging (MRI) models) is used to plan and carry out surgical procedures. However, exact registration of the patient imaging model in the AR system for providing sufficiently reliable AR interface for the surgeon is challenging for several reasons: (i) most of the patient's surface may be covered during surgery and is therefore not available for carrying out the registration, (ii) markers or stickers used in the prior art for triangulation do not provide sufficient accuracy, especially for deep and exact surgical procedures, (iii) using markers (e.g., fiducial markers) in the imaging process may require recent patient scanning and may not allow using past scans, and is often not sufficiently accurate. Also, it may leave the markers on the patient's body from scan to procedure, and (iv) changes in patient exact position and posture may be detrimental for the AR registration.

In contrast, disclosed embodiments provide device(s) that may function as registration template(s) that can be placed on the patient before the surgery, or during the surgery if re-adjustment of the AR registration is noted to be required. The AR system may be configured to identify the device and use its position to register the imaging model onto the AR model. The device may be rigid, made of sterilizable biocompatible material (e.g., sterilizable plastic), and may have features that relate to the specific patient, to the general patient anatomy and/or to the specific surgical procedure that is being carried out. The device may be 3D-printed ad hoc, or be used as a template for certain types of surgeries in which the anatomy is relatively similar among patients.

The specific shape of the device may be designed using the imaging model (and/or general anatomical data) and with reference to the type of surgery it is used for, to achieve maximal accuracy with respect to the geometrical characteristics of the surgical scene.

Advantageously, disclosed embodiments bridge between the real operational scene and the AR model using a physical device that is not used by prior techniques. Moreover, these technologies typically require that a significant portion of the patient is exposed, a condition which is typically not available during surgery, as the patient's body is typically mostly covered except for the very location of surgery. Advantageously, disclosed devices may provide patient-specific registration, in contrast to generic registration relating the real world directly to the virtual environment as taught by prior techniques, which lack the disclosed mediation of registration by the patient-related physical devices. An additional advantage of disclosed embodiments may include the avoidance of using fiducial markers and overcoming limitations associated with methods using them, included in prior techniques.

Advantageously, disclosed device embodiments may be non-invasive and indeed enable AR visualization that replaces the direct use of an invasive surgical guide that is taught by prior techniques. In disclosed embodiments, the registration of the device may be preparatory to the actual surgery and may not require using a surgical guide during the surgery and its registration to the head-mounted device as taught by prior techniques.

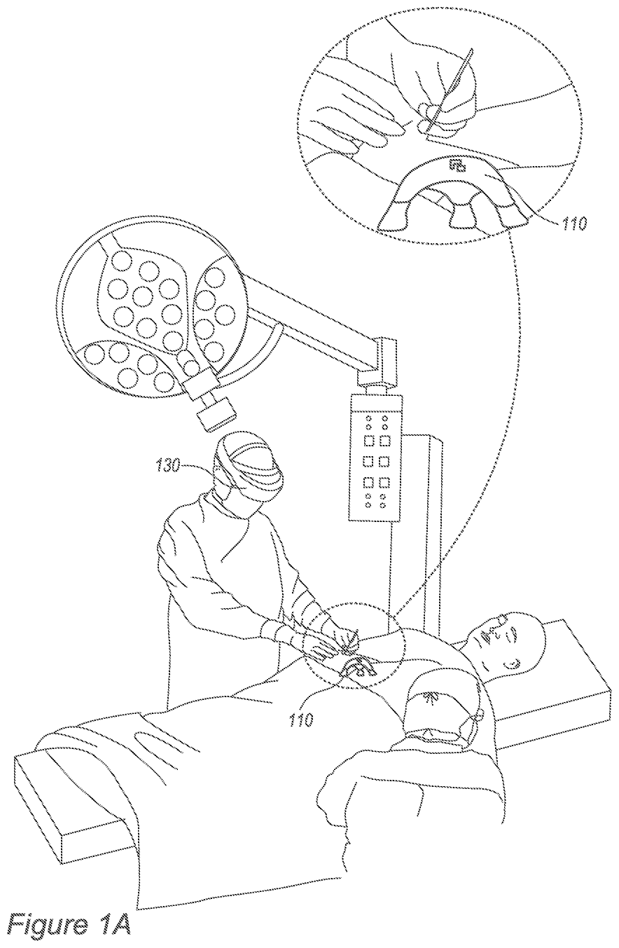

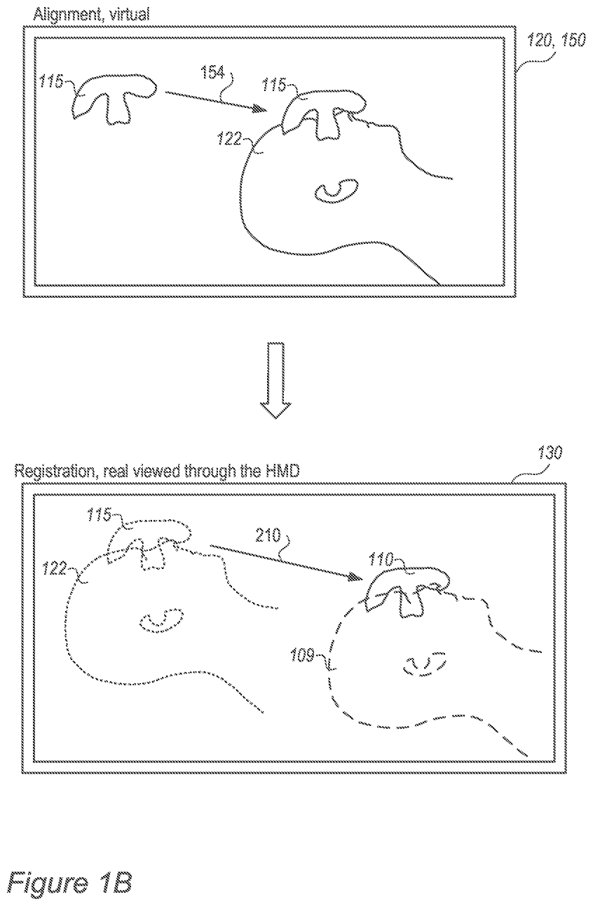

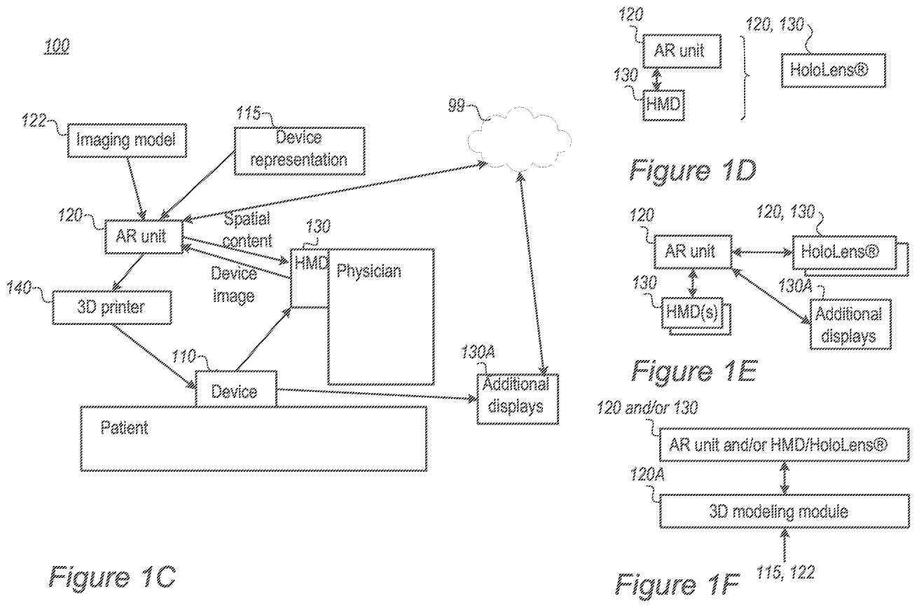

FIG. 1A is a high-level schematic illustration of an operation scene with a registration device 110, according to some embodiments of the invention; FIG. 1B is a high-level schematic illustration of using registration device 110, according to some embodiments of the invention; and FIG. 1C is a high-level schematic block diagram of registration system 100, according to some embodiments of the invention. System 100 may comprise an AR unit 120 comprising and/or in communication with a head mounted display (HMD) 130 used by a user, and a physical device 110 made of sterilizable biocompatible material and configured as a registration template. AR unit 120 and/or segmentation software 150 associated therewith may be configured to align a device representation 115 of physical device 110 onto an imaging model 122 of a patient, and AR unit 120 and/or HMD 130 may be configured to register, on HMD 130, device representation 115 with aligned imaging model 122 onto physical device 110, which is positioned with respect to the patient and is viewed through HMD 130—to display imaging model 122 or parts thereof in a corresponding spatial relation to the patient.

As illustrated schematically in FIG. 1A, one or more treating physicians and/or other personnel treating a patient and using HMD 130 may register patient-related spatial information (e.g., the position of the patient or of parts of the patient) to the actual scene of surgery using device 110 as reference that relates the patient-related spatial information to the actual morphological features of the patient being handled.

As illustrated schematically in FIG. 1B, an initial alignment step 154 on a virtual display associated, e.g., with AR unit 120 and/or with segmentation software 150, may be carried out by aligning device representation 115 onto patient's imaging model 122 (e.g., spatial data derived, e.g., from computerized tomography (CT), magnetic resonance imaging (MRI) and/or ultrasound (US) imaging). AR unit 120 may then spatially relate device representation 115 to imaging model 122, e.g., in form of a 3D combined model (e.g., as mesh or point cloud). Segmentation software 150 associated with AR unit 120 may be configured to carry out any data conversion involved, such as adjusting data representation methods and formats if needed. In various embodiments, the spatial relating of device representation 115 to imaging model 122 may be carried out automatically (e.g., by segmentation software 150 and/or AR unit 120), partly automatically (e.g., with manual verification) or manually. In various embodiments, the spatial relating of device representation 115 to imaging model 122 may be carried out by HMD 130 itself, automatically, partly automatically or manually.

During surgery, the user may only by required to carry out a registration step 210 that include moving (virtually) the combined model including device representation 115 spatially related to imaging model 122—so that device representation 115 overlaps actual physical device 110 as seen through HMD 130 of the user. Registration step 210 may be carried out with respect to the shape of physical device 110 with respect to device representation 115 and/or with respect to markings, patterns and/or codes (e.g., a QR code) on physical device 110. For example, HMD 130 may comprise software configured to or causing a processor to register device representation to physical device 110 morphologically, e.g., applying a transformation matrix to locate the virtual scene with respect to physical device 110. As imaging model 122 was aligned in step 154 to device representation 115—once device representation 115 is registered onto physical device 110, also imaging model 122 is correctly registered onto the actual patient (indicated by outline 109, e.g., as seen through HMD 130). For example, spatial content such as internal structures may be registered correctly with respect to the patient's anatomical features (e.g., onto which device 110 was placed). In various embodiments, registration 210 may be carried out automatically (e.g., by AR unit 120 and/or HMD 130), partly automatically (e.g., with manual verification) or manually.

In case multiple users with multiple HMDs 130 or other devices are present, any of them may perform registration step 210 independently—leading to parallel and specific registration of imaging model 122 onto the patient from the respective viewpoints, without need for further coordination and communication among HMDs 130 and between them and AR unit 120.

As illustrated schematically in FIG. 1C, various configurations of system 100 and AR unit 120 may be implemented. AR unit 120 may be integrated with HMD 130 and either AR unit 120 and/or HMD 130 may comprise a camera for capturing device 110 on the patient. For example, AR unit 120 and/or HMD 130 may comprise HMD and/or any type of AR device such as Microsoft® Hololens®. Any part of imaging model 122 and/or related medical information may be presented on HMD 130, as required during the surgery and according to the user's preferences. Additional HMDs and/or displays 130A may be in communication with AR unit 120, e.g., via communication link(s) 99 (e.g., WiFi, BlueTooth or any other communication protocol). In certain embodiments, additional HMDs and/or displays 130A may be co-registered to HMD 130 using only device 110 as common registration object, without use of any communication between the displays. Examples for additional HMDs and/or displays 130A may comprise additional HMDs (e.g., Hololens® devices) used by additional physicians present at the operation room and/or remote professionals, advisors, interns, trainees etc. Examples for additional HMDs and/or displays 130A may comprise other devices such as smartphones and/or remote displays used, e.g., for consulting, monitoring or teaching purposes in real-time. In certain embodiments, additional HMDs and/or displays 130A may comprise internal representations of the surgical system used by robotic surgical system(s) that may assist in the procedures or derive data relating to analysis of the procedures that are being carried out.

AR units 120 typically comprise a display surface or a projection module which provide additional content superimposed onto the user's field of view and/or onto content presented from a different source (e.g., a simulation or a model). For example, AR glasses may be used to add content onto the user's field of view, e.g., surgery-related data or images used to augment the operational scene viewed by the physician. AR unit 120 integrates the additional content with respect to the user's field of view and/or content from other sources. e.g., by registration which provide common spatial coordinates to the added content and the field of view and/or content from other sources (e.g., simulation or model). AR unit 120 may comprise various corresponding sensor(s) and communication module(s) to support the integration. Examples for AR units 120 include Microsoft® Hololens® and related devices, as well as any of eyeglasses mounted displays or projection units (e.g., virtual retinal display—VRD, or EyeTap), contact lenses, head-mounted displays (HMD), e.g., optical head-mounted displays (OHMD), heads-up display (HUD), as well as smartphone displays configured to provide AR content (e.g., content superimposed on content from the device's camera and/or from other sources). While AR unit 120 and HMD 130 are illustrated separately, they may be integrated as one device, possibly supported by linked processor(s).

FIGS. 1D-1F are high-level schematic block diagrams of AR units 120 and related HMDs 130, according to some embodiments of the invention. Various embodiments of AR units 120 and HMDs 130 or other HMDs and/or displays 130A may be used in various embodiments. For example, AR unit 120 may be separate from HMD(s) 130, or integrated therewith, e.g., in AR devices such as Microsoft® Hololens® systems. Hololens® systems may be used as independent device, without external AR unit 120 (see e.g., a schematic standalone example in FIG. 1D), or with AR unit 120, e.g., implemented by one or more processors to enhance the computational capacity of system 100 (see e.g., a schematic example in FIG. 1E). In some embodiments, AR unit 120 may be used as main processor that streams the AR content to HMD 130 and/or Hololens® 130, reducing the computational load thereupon. In some embodiments, multiple displays and/or HMDs 130, 130A are used, AR unit 120 may be configured to carry out the registration for all the user displays using the same physical device 110 (see e.g., a schematic example in FIG. 1E). In some embodiments, additional computational module(s) 120A (e.g., segmentation software 150) may be used to carry out at least part of the computational effort, e.g., a 3D modeling module 120A may be implemented separately or integrated with AR unit 120 to perform the combination of device representation 115 and anatomy imaging model 122, e.g., derived from imaging module or derived directly, e.g., via HMD 130 (see e.g., a schematic example in FIG. 1F).

For example, 3D modeling module 120A may comprise segmentation software 150 such as D2P™ (DICOM-to-PRINT, DICOM standing for the Digital Imaging and Communications in Medicine standard) for converting various types of medical data into 3D digital models (e.g., by converting slice data into volumetric data, applying image processing and/or AI algorithms for feature identification, etc.). Segmentation software 150 such as D2P™ may be configured to convert medical imaging data, e.g., CT, MRI and/or US images to any type of 3D model that can be processed digitally. For example, segmentation software 150 may convert imported DICOM images into respective 3D model(s) by segmenting the images and consolidating the segmentation to yield digital files which may be used in 3D printers, VR devices, surgical planning software and CAD software. AR unit 120 and/or segmentation software 150 may possibly be configured to apply image processing that is related to the specific procedure that is about to be perform, e.g., display only specified part(s) of imaging model 122 on HMD 130 according to the user's preferences. Device representation 115 may be imported into segmentation software 150 as a 3D model and/or as a virtual scan to be aligned with imaging model 122. Alignment 154 may be carried out via an interface to segmentation software 150 by applying movements and rotations to device representation 115 and/or imaging model 122 until they are aligned, possibly with respect to anatomical features that can be identified in imaging model 122. In certain embodiments, alignment 154 may be carried out at least partially automatically by segmentation software 150, e.g., with manual adjustments if required (in the virtual environment, see, e.g., FIG. 1B).

Alternatively or complementarily, HMDs 130 may be used to register device 110 directly with respect to the patient anatomy as viewed through HMD 130. In various embodiments, registration of device 110 may be carried out by any of AR unit 120, HMD 130 or by another device that communicates with AR unit 120 and/or HMD 130.

In various embodiments, physical device(s) 110 may be used to synchronize among multiple HMDs 130, such as multiple HoloLenses and/or multiple computer displays utilizing simple hardware (device(s) 110) rather than communication links. This approach may be especially beneficial in case of large data streams and communication loads instead of communicating complex 3D data, HMDs 130 may be synchronized by common registration of device 110. For example, imaging model 122 may comprise CT data, added information relating to the surgery and a model for the surgery—which can be very heavy computationally. Instead of prior art requiring streaming the data among HMDs 130, registration using device 110 may spare this requirement while providing full spatial synchronization among HMDs 130 and related units and modules. In case physical device 110 is required during surgery (e.g., if the patient moves, if the surgery includes multiple stages, etc.) simple reiteration of registration is achieved by placing physical device 110 at an appropriate position and spatially synchronizing HMDs 130 according to it. Even robotic systems may be spatially synchronized via device registration to replace or augment complex user interfaces for this purpose.

Imaging model 122 may be constructed from one or more sources, such as CT (computer tomography), MR (magnetic resonance data such as MRI—magnetic resonance imaging), US (ultrasound, e.g., when operating on stones, e.g., urinary or gall bladder stones), PET (positron emission tomography), etc. Imaging model 122 may be constructed directly using a 3D scanner generating a point cloud model that enables direct registration of device 110, with or without intermediate segmentation and generation of a mesh model. In certain embodiments, device 110 may be registered using external morphology only (e.g., when the anatomical features are prominent), without need for imaging data. In such cases, a 3D scanner may be used directly, without using an intermediate mesh model for the patient anatomy and/or for device 110. For example, external scanning only may be used for planning surgery procedures or for non-invasive procedure, or as baseline for invasive procedures, or for verification of older imaging data without requiring additional imaging (e.g., in urgencies or to reduce radiation applied to the patient).

In certain embodiments, imaging model 122 may at least partly comprise a virtual model, e.g., concerning circumferential areas or if no imaging data is available for the patient. It is emphasized that one of the advantages of disclosed devices 110 is that they may be used for registration even if specific imaging data is missing, and therefore do not necessarily require carrying out imaging procedures, or recent imaging data, in order to perform the registration. This advantage is significant in case of emergency operations, in cases where radiation avoidance is recommended, or if outdated imaging data is available that can serve as basis for the virtual model.

In various embodiments, disclosed registration may be used to carry out any of non-invasive procedures such as application of focused ultrasound, minimally invasive procedures and fully invasive procedures.

In certain embodiments, additional HMDs and/or displays 130A may comprise a simulation model used to check possible approaches to the surgery at hand. For example, a remote expert may use a simulation model that is spatially synchronized with the actual patient via registration of device 110 to check or verify different operational approaches and update the actual surgeon of preferred approaches. If needed, physical device 110 may be modified to provide better registration with respect to the suggested approach and be produced in real time at the operation room. While operating, the physician may still have model 110 displayed on HMD 130 (even when physical device 110 is removed) to help orient or navigate through the operation if needed and to maintain communication with the remote expert(s) with reference to physical device 110 if needed. Device 110 may be produced to include directive notations to assist the surgery. In certain embodiments, real collaboration during surgery may be enabled by physical device 110 in way that improves upon current patient specific simulations (e.g., as in a Procedure Rehearsal Studio™ system, PRS).

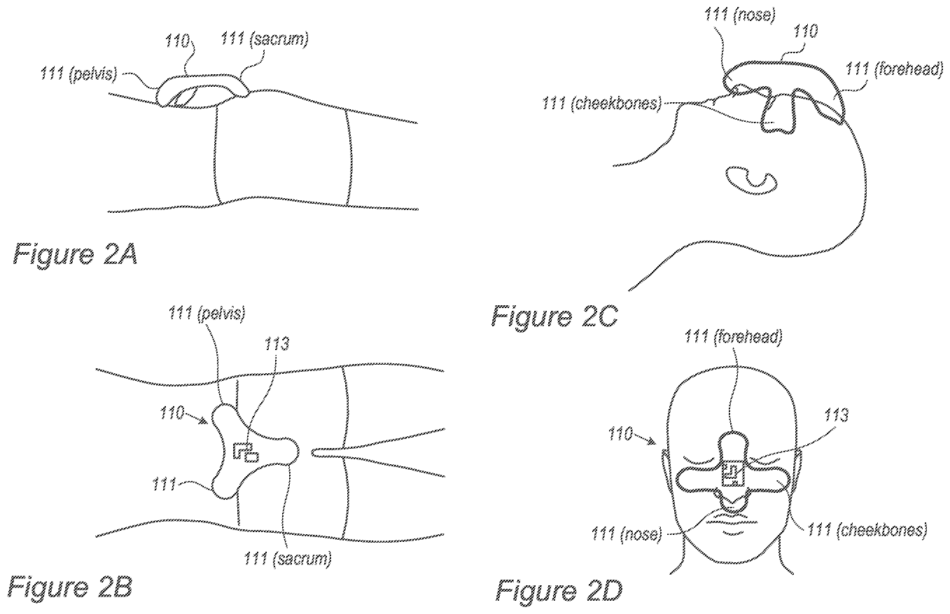

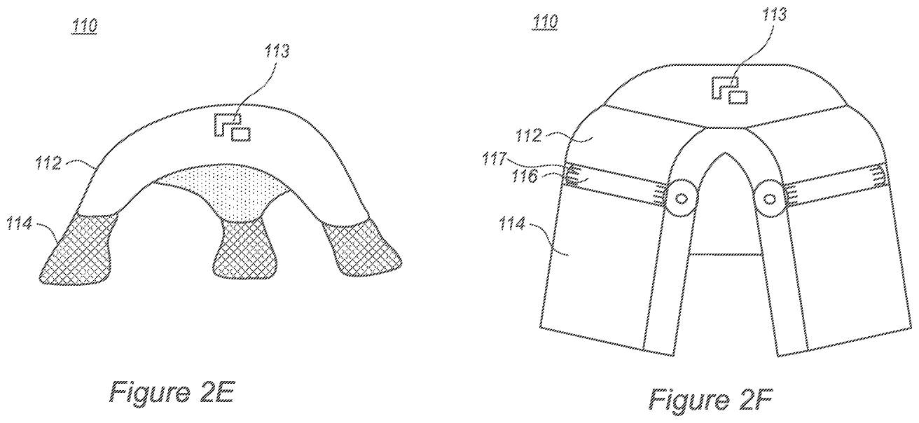

FIGS. 2A-2F include high-level schematic illustrations of physical devices 110, according to some embodiments of the invention. Physical devices 110 as registration templates may be shaped to fit to a specific patient, to a specified anatomical feature and/or to a specific surgical procedure.

FIGS. 2A and 2B illustrate schematically (in side and top views, respectively) physical device 110 placed above a patient's sacrum for surgery on the patient sacral region. Device 110 may be designed, as illustrated schematically, to have protrusions 111 that contact selected anatomical points of the patient, e.g., on the tops of posterior pelvis and sacral bones. Device 110 may further comprise markers 113 or other indications (e.g., coded graphics, stickers, trackers etc.) and/or have specific shapes or parts to assist registration.

FIGS. 2C and 2D illustrate schematically (in side and top views, respectively) physical device 110 placed above a patient's face for surgery on the patient facial region. Device 110 may be designed, as illustrated schematically, to have protrusions ill that contact selected anatomical points of the patient, e.g., the forehead, nose and cheekbones, as illustrated schematically. Device 110 may further comprise markers 113 or other indications (e.g., coded graphics, stickers, trackers etc.) and/or have specific shapes or parts to assist registration. Various embodiments of device 110 may be adjusted for use with respect to any of the patient's specific anatomical features or landmarks.

FIGS. 2E and 2F are high level schematic illustrations of physical devices 110, according to some embodiments of the invention. FIG. 2E provides a schematic example for devices 110 having two or more portions with different properties as disclosed herein, and FIG. 2F provides a schematic example for devices 110 having adjustable features for adapting device templates to specific patients, as disclosed herein.

It is noted that imaging model 122 may be configured to include parts that correspond to the patient's anatomical features onto which device 110 may be placed, such as facial bones (e.g., cheek, forehead), the nose or possibly teeth in the face, or bone protrusions of the pelvis or sacrum, as indicated schematically in FIGS. 2A-2D, or any other anatomical features.

Corresponding markers 113 may be used to designate individual device templates and/or device template parts in a distinguishable manner. In certain embodiments, AR unit 120 may be configured to detect unintentional changes or deviation in device 110 and provide corresponding alerts to prevent registration inaccuracies.

In certain embodiments, physical device 110 may have a first portion 112 used for the registration and a second portion 114 that is adjustable to specific patient characteristics and/or to changes in patient anatomy. For example, second portion 114 may comprise at least a part of a circumference of physical device 110 that is in contact with the patient. Second portion 114 may be flexible and/or be mechanically modifiable to yield the adjustment to the specific patient characteristics. For example, physical device 110 for facial surgery may have rigid first portion 112 and flexible circumference 114 (e.g., with portions 114 in FIG. 2E broadened to form a full circle or a part of a full circle or ellipse, or other circumferential form) for fitting onto a specific patient. In certain embodiments, device 110 may comprise a fixed upper-side geometry and an adjustable lower-side geometry, e.g., second portion 114 may be flexible and/or malleable (see, e.g., FIG. 2E). Device 110 may be produced from a template, e.g., from a library, and be adjusted digitally to the patient's anatomy and/or to changes in patient anatomy. Alternatively or complementarily, device 110 may be configurable at specific portions, such as joints 116 (see, e.g., FIG. 2F). For example, joints 116 may be cylindrical. The extent of deformation of one or more second portion 114 may be detected visually, e.g., by AR unit 120 and/or HMD 130, and optionally graduation marks 117 may be used to indicate directly the extent of deformation or modification (e.g., rotation) applied to portions of device 110 in adapting them to specific patient's anatomical features.

In various embodiments, device 110 may comprise various coloration and/or patterns to provide or assist efficient and accurate optical registration. In various embodiments, device 110 may comprise multiple separate (or interconnected) parts as multiple devices 110 used simultaneously for registration. For example, in case one big (e.g., few tens of cm wide) device 110 would be inconvenient for use, smaller and possibly multiple devices 110 may be set on anatomical features, and registration may be carried out with respect to multiple devices 110. Alternatively or complementarily, adjusting registration may be carried out by using or adding small device(s) 110, e.g., during operation to enhance the accuracy of the registration. In certain embodiments, device 110 may be placed outside the direct region of surgery, possibly on remoter anatomical landmarks or even placed beside the patient to provide registration with less obstruction and/or with respect to more prominent landmarks than available in the direct proximity of the location that is operated upon. Possibly, when device 110 is used without contacting the patient, it may be sterilized less frequently and/or with other operation room equipment, or be re-used during the operation without additional sterilization, Different types of device 110 may be used under different circumstances such as stage of the operation and the required and achieved registration accuracy and may be switched to accommodate for changing circumstances.

Devices 110 may be produced as modifiable templates, e.g., for different patient characteristics such as size, age, anatomical features, etc., and by modified to fit a specific patient upon demand. In certain embodiments, specific device modifications may be provided together with the device template(s) as instructions for adjustment of the template(s) to specific situation, e.g., specific rotation angles may be suggested for specific uses. Adjustable devices 110 may be 3D-printed as one piece and/or as multiple pieces that can be assembled to form device 110. AR unit 120 and/or HMD 130 may be configured to identify graduation marks 117 and determine therefrom the exact structure of modified device template 110.

In certain embodiments, system 100 may comprise one or more libraries of device representations 115, as specific devices and/or as templates, to be selected from prior to specific operations. For example, codes may be used to designate device representations 115 and relate them to specific operations, anatomical regions, patient characteristics and/or specific patients. Correspondingly, simulations and/or reconstructions of specific procedures may be enabled in relation to the registration devices used therein.

In certain embodiments, device 110 may comprise one or more large re-usable part(s) (e.g., portion 112) and small adjustable disposable parts (e.g., portions 114) to reduce time and material required for printing in specific cases.

Device 110 may be customized to a specific patient, to a specific operation and/or to specific anatomical features. In certain embodiments, the shape of device 110 may be defined using imaging model 122 of the patient, configuring the shape to fit a portion of the patient's body that is close to the location of surgery (e.g., in oral and maxillofacial surgery). Corresponding device 110 may be printed by 3D printer 140 (in one or more copies) as part of the preparation procedure for the surgery, or even during surgery, once a 3D printer that is quick enough is available, e.g., if modifications are found to be required, or if copies are needed. Alternatively or complementarily, one or more adjustable device templates 110 may be used for providing and/or supplementing device 110.

Alternatively, or complementarily, device 110 may be prepared in advance according to specified anatomical feature, such as the anatomical regions of the sacrum, sternum or other relatively stable structures. Devices 110 may comprise parts which can be fine-tuned to the exact anatomy of the patient if needed, either by physical manipulation of device 110 (e.g., removing parts, pressing flexible parts etc.) or by modifying a device template upon actual printing the device as preparation for surgery on a specific patient. Devices 110 may also be shaped for internal use, e.g., in case of major operational intervention for enhancing the accuracy of registration for inner organs or implants.

In certain embodiments, AR unit 120 may be further configured to derive a shape of physical device 110 from imaging model 122 and/or from specific patient characteristics of a specific patient. In certain embodiments, AR unit 120 may be configured to adjust a given template for physical device 110 according to specific patient characteristics, and send the adjusted template to 3D printer 140 for printing a personalized physical device 110.

In any of the embodiments, 3D printer 140, e.g., adjacent to the operation room, may be configured to print physical device 110 in preparation for and/or during surgery according to a given template and/or according to an adjusted template provided by AR unit 120.

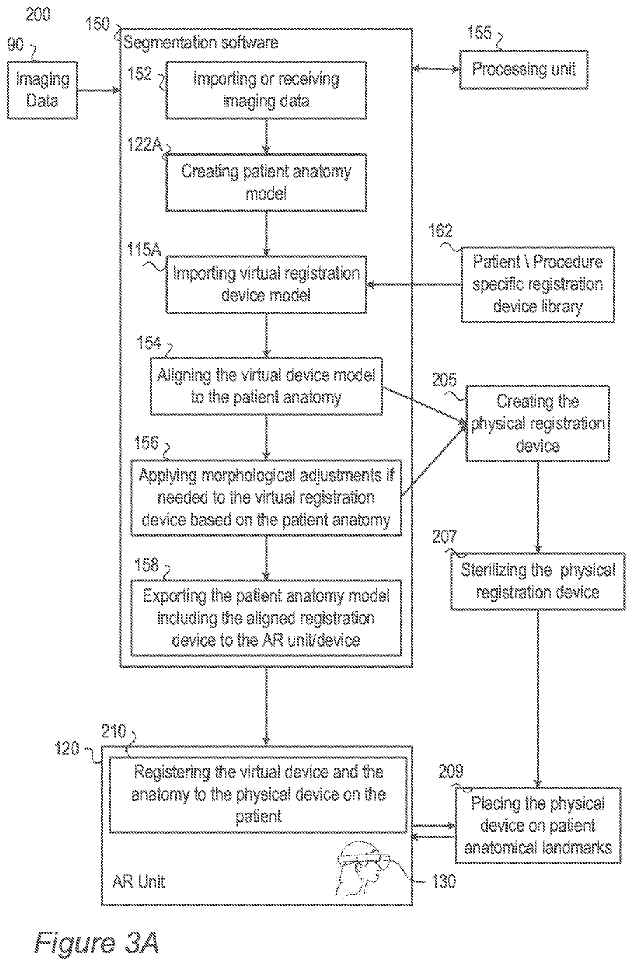

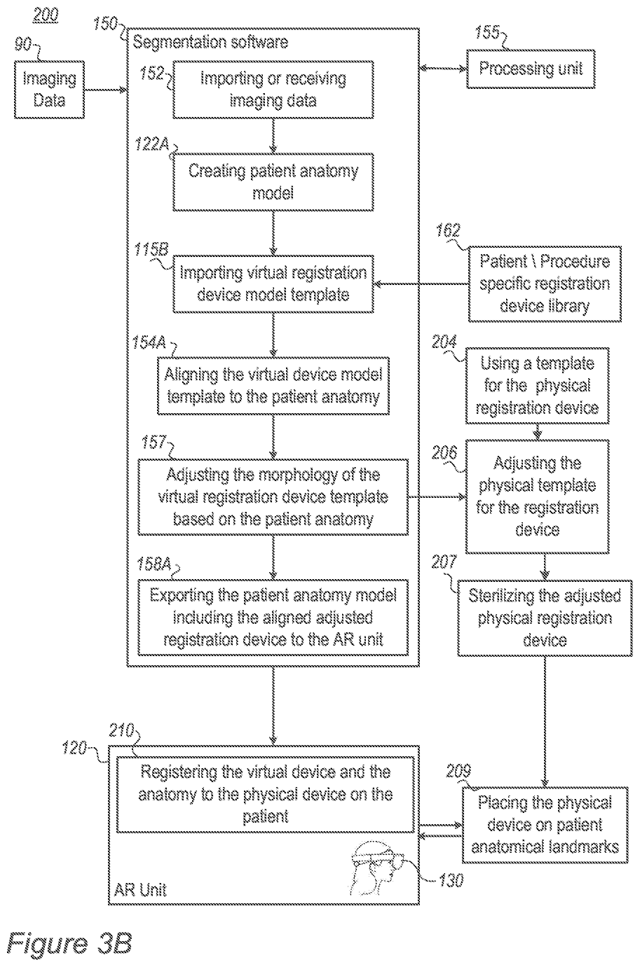

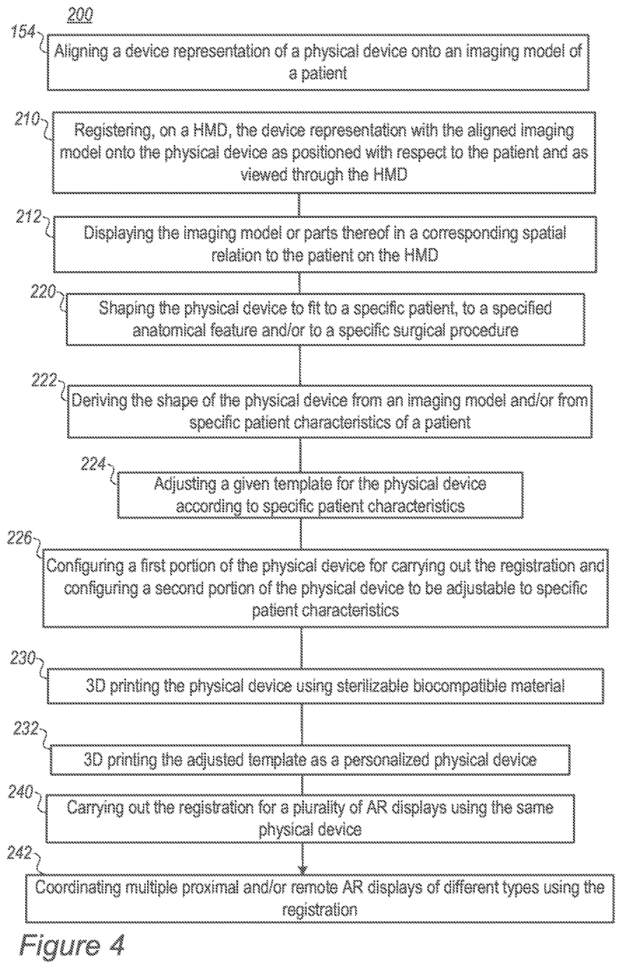

FIGS. 3A, 3B and 4 are high-level flowcharts illustrating methods 200, according to some embodiments of the invention. Method 200, as illustrated schematically in FIG. 3A, may comprise using imaging data 90 as initial input for segmentation software 150 which may be at least partly implemented in AR unit 120 (possibly even within HMD 130) and/or may be at least partly implemented in a processing unit 155. Method 200, as implemented in segmentation software 150, may comprise importing or receiving imaging data 90 (stage 152), e.g., data of or describing a patient or a portion of a patient, creating the patient anatomy model 122 (stage 122A), e.g., using segmentation software such as D2P™ described above and/or possibly by applying additional image processing, e.g., supported by artificial intelligence (AI) or deep learning methodologies to extract specified features, importing (or generating) the virtual registration device model of physical device 110, which may corresponding to device representation 115 (stage 115A), e.g., from a patient- and/or procedure-specific registration device library 162, aligning the virtual device model to the patient anatomy (stage 154), optionally followed by creating 205 (e.g., by 3D printing) physical registration device 110 and sterilizing 207 the device, applying morphological adjustments if needed to the virtual registration device based on the patient anatomy (stage 156) and exporting the patient anatomy model including the aligned registration device to the AR unit/device 130/120, respectively (stage 158). Once the device model is adjusted to the patient and/or the procedure and is produced, the physical device may be placed in on patient anatomical landmarks (stage 209) and registered with the virtual device and the anatomy in AR unit 120 and/or HMD 130. It is noted that AR unit 120 and/or HMD 130 may further be used to adjust the placement of physical device 110 if needed, e.g., to improve registration, increase proximity to the regions of interest etc.

Method 200, as illustrated schematically in FIG. 3B, may comprise using a template for the physical registration device (stage 204), for example, an adjustable, flexible and/or malleable device template, at one or more sizes, that can be adjusted to specific patients by deformation, relative movements of its parts and/or abrasion or removal of template parts according to specific patient characteristics as disclosed herein. Following an importation of the virtual registration device model template (stage 115B) and an alignment of the virtual device model template to the patient anatomy (stage 154A), the morphology of the virtual registration device template may be adjusted based on the patient anatomy (stage 157) and the physical template for the registration device may be adjusted accordingly (stage 206), followed by sterilization 207 and by exporting of the patient anatomy model including the aligned adjusted registration device (template) to the AR unit (stage 158A). The device template and its adjustment may be carried out with respect to one or more device templates (e.g., having different sizes and/or proportions of parts) and/or with respect to one or more device template portions. Corresponding markers 113 may be used to designate individual device templates and/or device template parts in a distinguishable manner.

Example method 200, as illustrated schematically in FIG. 4, may comprise aligning a device representation of a physical device onto an imaging model of a patient (stage 154), and registering, on a HMD, the device representation with the aligned imaging model onto the physical device as positioned with respect to the patient and as viewed through the HMD (stage 210)—to display the imaging model or parts thereof in a corresponding spatial relation to the patient on the HMD (stage 212).

Registration 210 may be carried out by various embodiments to ensure spatial correspondence in the virtual environment for data from different sources, such imaging model 122 and/or parts thereof and the actual patient as viewed though, e.g., HMD 130. Registration 210 may comprise spatial transformations between data sets that are represented in space, e.g., from device representation 115 to physical device 110 as imaged in HMD 130 to verify they coincide. Corresponding spatial transformations may be applied to imaging model 122 as those required to transform device representation 115 to physical device 110, so that imaging model 122 is registered onto the patient. The spatial transformations may relate different coordinate systems, and may depend on the formats and spatial characteristics of the data sets, and on possible modifications or adjustments of physical device 110 as disclosed herein. For example, the spatial transformations may relate to changes in the viewing angle and distance to physical device 110 and/or spatial characteristics of imaging model 122. In various embodiments, registration algorithms that are part of the application programming interface (API) of HMD 130 (e.g., Hololens®) may be used to perform registration 210.

In various embodiments, method 200 may comprise for example, any of the following stages: shaping the physical device to fit to a specific patient, to a specified anatomical feature and/or to a specific surgical procedure (stage 220); deriving a shape of the physical device from an imaging model and/or from specific patient characteristics of a patient (stage 222); and/or adjusting a given template for the physical device according to specific patient characteristics (stage 224), and 3D printing the adjusted template as a personalized physical device (stage 232). In certain embodiments, method 200 may comprise configuring a first portion of the physical device for carrying out the registration and configuring a second portion of the physical device to be adjustable to specific patient characteristics (stage 226).

In any of the embodiments, method 200 may further comprise carrying out the registration for a plurality of AR displays using the same physical device (stage 240) and/or coordinating multiple proximal and/or remote AR displays of different types using the registration (stage 242).

Physical device 110 may be made of a range of sterilizable biocompatible materials. In some embodiments, physical device 110 may be produced by various procedures, possibly other than 3D printing, and may be made of any sterilizable biocompatible material, including various polymers (e.g., nylon), plastics or metals (e.g., titanium). In certain embodiments, physical device 110 may be produced using 3D printing, possibly using 3D printer(s) adjacent to the operation room and providing device(s) 110 as part of the preparation to the surgery or even during surgery if need arises. In case physical device 110 is 3D printed, it may be made from corresponding compatible materials, which are also sterilizable and biocompatible. Non-limiting examples include materials that can be used with 3DSystems® Figure 4® printers such as Figure 4® MED-WHT 10 which is a rigid white material and Figure 4® MED-AMB 10 which is a rigid translucent material—both being UV-cured polymers which are biocompatible and sterilizable. Other materials may comprise polymers such as ABS (acrylonitrile butadiene styrene) or modifications thereof, or any other plastic materials—as long as they are biocompatible and sterilizable. Additional examples for materials comprise DuraForm PA (SLS) which is a durable thermoplastic with balanced mechanical properties and fine-features surface resolution, or other nylon-like and/or polypropylene-like thermoplastics that are biocompatible and sterilizable. Any of these materials may be cured, e.g., by UV or laser, as part of the device's production process. Metals such as titanium or alloys thereof may also be used to produce physical device 110.

The inventors note that using 3D-printable material is advantageous in terms of required time to prepare devices 110 before the surgery, avoiding waste of operation room time. For example, using pre-prepared template with automatic segmentation may allow 3D printing device 110 upon requirement within 1-1.5 hours, including sterilization, and can be used in surgical planning without any time penalty.

Devices 110 may comprise flexible material, a combination of rigid and flexible materials, or rigid material, and may include markers (e.g., titanium markers) and/or stickers for assisting registration. Part(s) of device 110 may be adjustable (e.g., by being flexible or modifiable, e.g., by cutting or curving of edges, or using Boolean operations for exact adjustment) to patient's surface features, while specific part(s) of device 110 may be configured to simplify registration. Boolean operations such as subtraction, intersection, addition, uniting etc. in the context of CAD (Computer-aided design) operation may be applied to adjust a part of the device template to the exact patient features as derived from a scanning of the patient and/or imaging model 122, e.g., by subtracting the scanned or modeled features from a region of the virtual device template (to yield adjusted virtual device representation 115). Following the adjustments, device 110 may be 3D printed to fit the specific patient features, e.g., as preparation for a surgery. Specific device features may be left unchanged for registration purposes and/or AR unit 120 may register physical device 110 according to adjusted virtual device representation 115.

In certain embodiments, multiple devices 110 may be prepared, as alternative designs or as complementary devices for different stages of surgery, for verifying or re-establishing registration if needed. It is noted that 3D printers allow preparing multiple devices 110 for multiple surgeries, simultaneously.

Advantageously, simple device 110 and simple use of device 110 may spare expensive time during the surgical procedure and allow reaching maximal registration accuracy before and during surgery. For example, using simple physical model device 110 for registration and adjustments is simpler than using gestures to place a virtual model at the right position with respect to the patient. The simplicity of use enables adjusting to changes during surgery by re-application of physical device 110 and adjusting registration accordingly. Physical device 110 thus provides a physical user interface for the surgeon to adjust AR registration during operation if needed (e.g., following patient movements or position adjustments).

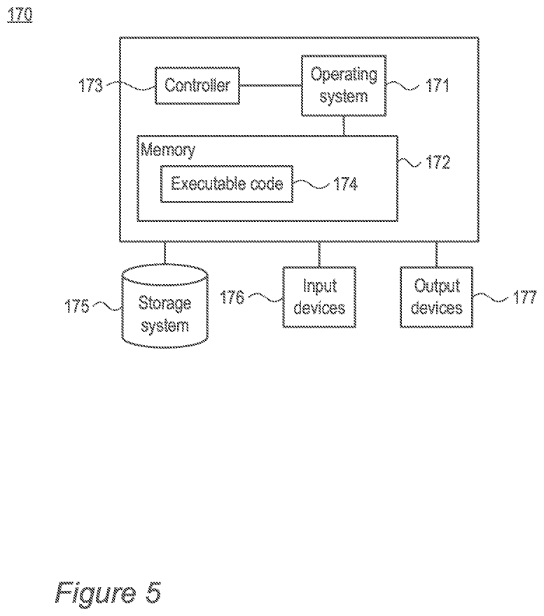

FIG. 5 is a high-level block diagram of an exemplary computing device 170, which may be used with embodiments of the present invention. For example, computing device 170 may be used, at least on part, to implement at least one of AR unit 120, HMD 130 and/or deriving and processing imaging model 122 and/or device representation 115. Additionally or complementarily, processing unit 155 and/or segmentation software 150 may be at least partly implemented by computing device 170 or part(s) thereof.

Computing device 170 may include a controller or processor 173 that may be or include, for example, one or more central processing unit processor(s) (CPU), one or more Graphics Processing Unit(s) (GPU or general-purpose GPU—GPGPU), a chip or any suitable computing or computational device, an operating system 171, a memory 172, a storage 175, input devices 176 and output devices 177.

Operating system 171 may be or may include any code segment designed and/or configured to perform tasks involving coordination, scheduling, arbitration, supervising, controlling or otherwise managing operation of computing device 170, for example, scheduling execution of programs. Memory 172 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory 172 may be or may include a plurality of possibly different memory units. Memory 172 may store for example, instructions to carry out a method (e.g., code 174), and/or data such as user responses, interruptions, etc.

Executable code 174 may be any executable code, e.g., an application, a program, a process, task or script. Executable code 174 may be executed by controller 173 possibly under control of operating system 171. For example, executable code 174 may when executed cause the production or compilation of computer code, or application execution such as VR execution or inference, according to embodiments of the present invention. Executable code 174 may be code produced by method embodiments described herein. For the various modules and functions described herein, one or more computing devices 170 or components of computing device 170 may be used. Devices that include components similar or different to those included in computing device 170 may be used, and may be connected to a network and used as a system. One or more processor(s) 173 may be configured to carry out embodiments of the present invention by for example executing software or code.

Storage 175 may be or may include, for example, a hard disk drive, a floppy disk drive, a Compact Disk (CD) drive, a CD-Recordable (CD-R) drive, a universal serial bus (USB) device or other suitable removable and/or fixed storage unit. Data such as instructions, code, VR model data, parameters, etc. may be stored in a storage 175 and may be loaded from storage 175 into a memory 172 where it may be processed by controller 173. In some embodiments, some of the components shown in FIG. 5 may be omitted.

Input devices 176 may be or may include for example a mouse, a keyboard, a touch screen or pad or any suitable input device. It will be recognized that any suitable number of input devices may be operatively connected to computing device 170 as shown by block 176. Output devices 177 may include one or more displays, speakers and/or any other suitable output devices. It will be recognized that any suitable number of output devices may be operatively connected to computing device 170 as shown by block 177. Any applicable input/output (I/O) devices may be connected to computing device 170, for example, a wired or wireless network interface card (NIC), a modem, printer or facsimile machine, a universal serial bus (USB) device or external hard drive may be included in input devices 176 and/or output devices 177.

Embodiments of the invention may include one or more article(s) (e.g., memory 172 or storage 175) such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein.

Aspects of the present invention are described above with reference to flowchart illustrations and/or portion diagrams of methods, apparatus (systems) and computer program products according to embodiments of the invention. It will be understood that each portion of the flowchart illustrations and/or portion diagrams, and combinations of portions in the flowchart illustrations and/or portion diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or portion diagram or portions thereof.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or portion diagram or portions thereof.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or portion diagram or portions thereof.

The aforementioned flowchart and diagrams illustrate the architecture, functionality, and operation of possible implementations of systems, methods and computer program products according to various embodiments of the present invention. In this regard, each portion in the flowchart or portion diagrams may represent a module, segment, or portion of code, which comprises one or more executable instructions for implementing the specified logical function(s). It should also be noted that, in some alternative implementations, the functions noted in the portion may occur out of the order noted in the figures. For example, two portions shown in succession may, in fact, be executed substantially concurrently, or the portions may sometimes be executed in the reverse order, depending upon the functionality involved. It will also be noted that each portion of the portion diagrams and/or flowchart illustration, and combinations of portions in the portion diagrams and/or flowchart illustration, can be implemented by special purpose hardware-based systems that perform the specified functions or acts, or combinations of special purpose hardware and computer instructions.

In the above description, an embodiment is an example or implementation of the invention. The various appearances of “one embodiment”, “an embodiment”. “certain embodiments” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment. Certain embodiments of the invention may include features from different embodiments disclosed above, and certain embodiments may incorporate elements from other embodiments disclosed above. The disclosure of elements of the invention in the context of a specific embodiment is not to be taken as limiting their use in the specific embodiment alone. Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described. Meanings of technical and scientific terms used herein are to be commonly understood as by one of ordinary skill in the art to which the invention belongs, unless otherwise defined. While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claimed is:

1. A system comprising:
an augmented reality (AR) unit comprising or in communication with a head mounted display (HMD) used by a user, and
a physical device made of sterilizable biocompatible material and configured as a registration template,
wherein the AR unit or segmentation software associated therewith is configured to align a device representation of the physical device onto an imaging model of a patient, and
wherein the AR unit or the HMD is configured to register, on the HMD, the device representation with the aligned imaging model onto the physical device, which is positioned with respect to the patient and is viewed through the HMD, to display the imaging model or parts thereof in a corresponding spatial relation to the patient.

2. The system of claim 1, wherein the physical device is shaped to fit to a specific patient, to a specified anatomical feature and/or to a specific surgical procedure.

3. The system of claim 1, wherein the AR unit is further configured to derive a shape of the physical device from an imaging model and/or from specific patient characteristics of a patient.

4. The system of claim 1, wherein the AR unit is further configured to adjust a given template for the physical device according to specific patient characteristics and send the adjusted template to a 3D printer for printing a personalized physical device.

5. The system of claim 1, wherein the physical device has a first portion used for the registration and a second portion that is adjustable to specific patient characteristics.

6. The system of claim 5, wherein the second portion comprises at least a part of a circumference of the physical device that is in contact with the patient.

7. The system of claim 6, wherein the second portion is flexible and/or mechanically modified to yield the adjustment to the specific patient characteristics.

8. The system of claim 1, further comprising a 3D printer configured to print the physical device in preparation for and/or during surgery according to a given template and/or according to an adjusted template provided by the AR unit.

9. The system of claim 1, further comprising a plurality of user displays, and wherein the AR unit is configured to carry out the registration for all the user displays using the same physical device.

10. The system of claim 9, wherein the user displays are independently selected from: an AR device, a different HMD, a smartphone, a remote display, and an internal representation of the surgical system used by a robotic surgical system.

11. A method comprising:
aligning a device representation of a physical device onto an imaging model of a patient, and
registering, on a HMD, the device representation with the aligned imaging model onto the physical device as positioned with respect to the patient and as viewed through the HMD, to display the imaging model or parts thereof in a corresponding spatial relation to the patient on the HMD.

12. The method of claim 11, further comprising 3D printing the physical device using sterilizable biocompatible material.

13. The method of claim 11, further comprising shaping the physical device to fit to a specific patient, to a specified anatomical feature and/or to a specific surgical procedure.

14. The method of claim 11, further comprising deriving a shape of the physical device from the imaging model and/or from specific patient characteristics of a patient.

15. The method of claim 11, further comprising adjusting a given template for the physical device according to specific patient characteristics, and 3D printing the adjusted template as a personalized physical device.

16. The method of claim 11, further comprising configuring a first portion of the physical device for carrying out the registration and configuring a second portion of the physical device to be adjustable to specific patient characteristics.

17. The method of claim 11, further comprising carrying out the registration for a plurality of AR displays using the same physical device.

18. The method of claim 17, further comprising coordinating multiple proximal and/or remote AR displays of different types using the registration.

19. A computer program product comprising a non-transitory computer readable storage medium having computer readable program embodied therewith, the computer readable program configured to carry out the method comprising:
for a device representation of a physical device aligned onto an imaging model of a patient:
registering, on a HMD, the device representation with the aligned imaging model onto the physical device as positioned with respect to the patient and as viewed through the HMD, to display the imaging model or parts thereof in a corresponding spatial relation to the patient on the HMD.

20. The computer program product of claim 19, wherein the physical device is printed using sterilizable biocompatible material.
